(19) United States
(12) Patent Application Publication
(10) Pub. No.: US 2020/0350070 A1
(43) Pub. Date: Nov. 5, 2020

(54) SYSTEM AND METHOD FOR DETERMINATION AND CONCURRENT DISPLAY OF MEDICAL EXPERT OPINION AND GUIDELINES

(71) Applicant: WebMD, LLC, New York, NY (US)

(72) Inventors: Helen Moran, Hastings-on-Hudson, NY (US); Jason Penchoff, New York, NY (US); Jo-Ann Strangis, New York, NY (US); Lisa Greenbaum, Nashville, TN (US); Christopher Michaud, Brooklyn, NY (US); Melissa Karame, Pelham, NY (US)

(21) Appl. No.: 16/403,310

(22) Filed: May 3, 2019

(51) Int. Cl. G16H 50/20; G16H 70/20; G06F 3/14

(52) U.S. Cl. CPC G16H 50/20; G06F 3/0482; G06F 3/14; G16H 70/20

(57) ABSTRACT

A method of determining and causing display of particularized medical information is disclosed. The method comprises operations of receiving user input corresponding to a set of parameters indicating a medical condition and parsing the set of parameters to determine one or more identifiers corresponding to the medical condition. Additional operations of the method include querying an expert opinion database according to the one or more identifiers to retrieve an expert opinion entry corresponding to the medical condition and querying a guideline excerpt database according to metadata of the expert opinion entry to retrieve a guideline excerpt corresponding to the expert opinion. Following the querying of the expert opinion database and the guideline excerpt database, the method includes the operation of transmitting instructions to a network device to cause rendering of a display illustrating the video file and one of the transcript of the video file or the guideline excerpt.

### FIELD

Embodiments of the disclosure relate to the field of text generation and display. More specifically, one embodiment of the disclosure relates to a system and method for determining and automatically displaying medical data including expert opinions, transcripts thereof and corresponding medical guideline section excerpts at a granular clinical level. Further, one embodiment of the disclosure relates to a system and method of determining and automatically displaying oncology data including expert opinion excerpts, transcripts and corresponding medical guideline sections via a dynamic graphical user interface (GUI).

### GENERAL BACKGROUND

Medical professionals constantly refer to professional medical guidelines when diagnosing a patient and when determining a treatment plan. In fact, several professional medical guidelines may cover a single medical field. For example, the National Comprehensive Cancer Network (NCCN) guidelines, the American Society of Clinical Oncology (ASCO) guidelines and the European Society for Medical Oncology (ESMO) guidelines are just a few examples that apply to oncology. Therefore, a medical professional may spend precious time searching through multiple professional guidelines, which are each often hundreds or thousands of pages long.

Additionally, as the process for content being published within a professional guideline may take months, if not years, the professional guidelines are typically lacking the most up-to-date information. This information may be a result of trials or discoveries that occur at either universities and/or research medical centers affiliated with a hospital and not be widely known or disseminated until publication in a professional guideline. Specifically, this information may be needed to develop a treatment plan that will provide the highest likelihood of survival for a patient.

Therefore, what is needed is a system and method that provides quick and easy access to information of a specific and particularized area of medicine that includes both relevant sections of one or more medical guidelines as well as relevant expert opinions relating to clinical trial results and new discoveries that may be too new to be included in the professional guidelines.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 is an exemplary system diagram illustrating communications between a decision point system and a plurality of network devices according to some embodiments;

FIG. 2 is an exemplary embodiment of logical representations of the decision point system operating in cloud computing services and a decision point mobile application operating on a mobile device according to some embodiments;

FIG. 3 is a flowchart illustrating a process of determining one or more expert opinions and one or more guideline excerpts to be rendered on a display screen as performed by the decision point system according to some embodiments;

FIG. 4 is a flowchart illustrating a first embodiment of a detailed process of determining one or more expert opinions and one or more guideline excerpts to be rendered on a display screen as performed by the decision point system;

FIG. 5 is a flowchart illustrating a second embodiment of a detailed process of determining one or more expert opinions and one or more guideline excerpts to be rendered on a display screen as performed by the decision point system; and

FIGS. 6A-6F are illustrations of display screens an exemplary embodiment of the detailed process of FIG. 4.

### DETAILED DESCRIPTION

Various embodiments of the disclosure are directed to a system and a method for performing a process that includes receiving user input via a dynamic graphical user interface (GUI), responsive to the user input filtering through a content within a library database in order to dynamically update the GUI until a determination is made regarding display of content that may be related to various branches of medicine including oncology. The content to be displayed may include content related to a specific and particularized area of oncology (e.g., a cancer type, a tumor, a particular cancer stage, etc.). The format of the displayed content may include one or more parsed medical guidelines and videos corresponding to expert opinions of the specific and particularized area of oncology. Additionally, the displayed content may include a text transcript of the audio provided in the video of the expert opinion. Examples of medical guidelines include, but are not limited or restricted to, the National Comprehensive Cancer Network (NCCN) guidelines, the American Society of Clinical Oncology (ASCO) guidelines, the European Society for Medical Oncology (ESMO) guidelines.

As user input is received via the GUI, the system automatically filters curated content stored within one or more data stores and dynamically updates the GUI. The updated GUI may provide either additional filtering options and/or options for selecting viewable content (e.g., selectable tabs related to a specific and particularized area of oncology). Responsive to receiving a selection of specific and particularized area of oncology, the system automatically retrieves from one or more data stores, in one instance, (i) a video of an expert opinion corresponding to that specific and particularized area of oncology, (ii) a transcript of the video, and (ii) one or more portions of guidelines corresponding to that specific and particularized area of oncology. The system then causes one or more of (i)-(iii) to be displayed on a display screen of the electronic of the user enabling quick access to the most recent information on the desired specific and particularized area of oncology.

I. Overview

As described below, an automated system (referred to as a “decision point system”) is configured to quickly and efficiently enable a medical professional, patient, or other user, to access medical information related to a particularized medical condition. As one illustrative embodiment, the decision point system may operate within cloud computing services and be accessible by a doctor via a mobile application (“the decision point mobile application”) and/or via loading a website with an internet browser. In either instance, the decision point system is configured to provide instructions for rendering a displaying that enables the doctor to provide input to specify a particular medical condition according to guided questions. For example, a doctor may provide input indicating a tumor type being colorectal cancer followed by input indicating a cancer type being adenocarcinoma. The decision point system may subsequently analyze decision pathway data and provide, via either the decision point mobile application or the website, additional options for providing user input. The additional options correspond directly to the previously received input (e.g., tumor type and cancer type). In some embodiments, the decision pathway data may include a predetermined hierarchical data structure, such as data stored in as a structure of nodes and dependencies (e.g., a tree structure, or other structure having more than one root node). In other embodiments, the decision pathway data may correspond to one or more predetermined rule sets. In yet other embodiments, the decision pathway data may correspond to a machine learning model that is configured to be applied to received user input. The decision point system continues to receive user input and provide additional options with each round of input received further specifying or narrowing the medical condition for which the doctor wishes to have information (“medical condition at issue”).

In some instances, the decision point system may provide the doctor with information as well as options for providing more information. For instance and continuing the example above, after receiving input corresponding to a tumor type being colorectal cancer followed by input indicating a cancer type being adenocarcinoma, the decision point system may receive additional information (i.e., selection of one of a set parameters as determined by the decision point system according to analysis of the decision pathway data) such as a tumor extent being T3. In some embodiments, the decision point system may then provide (1) additional options for receiving more information to narrow the medical condition at issue, and (2) a set of expert opinions and/or professional medical guideline excerpts that apply to the user input received thus far. In other embodiments, the set of expert opinions and/or professional medical guideline excerpts may be provided upon a determination by the decision point system that the medical condition has been narrowed to the full extent of the decision pathway data (e.g., traversal of the tree structure to a node having no children based on user input).

According to some embodiments of the disclosure, a method of determining and causing display of particularized medical information is disclosed. In particular, the method comprises operations of receiving user input corresponding to a set of parameters indicating a medical issue and parsing the set of parameters to determine one or more identifiers corresponding to the medical issue. Further, subsequent to determining one or more identifiers corresponding to the medical issue, additional operations of the method include querying an expert opinion database according to the one or more identifiers to retrieve a first expert opinion entry corresponding to the medical issue, wherein the first expert opinion entry includes (i) a video file, (ii) a transcript of the video file and (iii) metadata. In some embodiments, an additional operation of the method includes querying a guideline excerpt database according to the metadata to retrieve a first guideline excerpt corresponding to the first expert opinion. In some embodiments of the method, the metadata indicates a link between the first expert opinion entry and one or more guideline excerpts including the first guideline excerpt. In one exemplary embodiment, each guideline excerpt, and any information associated therewith, may be stored within a separate table within a content management system (CMS). Further, each table within the CMS may be linked to a particular identifier.

Following the querying of the expert opinion database and the guideline excerpt database, the method may include the operation of transmitting instructions to a network device to cause rendering of a display illustrating the video file and one of the transcript of the video file or the first guideline excerpt. In some embodiments, the medical issue may correspond to cancer, and the set of parameters may be indicative of a medical diagnosis of the cancer. In additional detail, in some embodiments, examples of parameters includes, but are not limited or restricted to, a tumor type, a cancer type, a cancer stage, a molecular test indicator, a tumor extent indicator, a tumor stage, a metastasis level indicator, and a biomarker.

In yet other embodiments, the method includes an additional operation of, responsive to receiving initial user input that includes the tumor type and the cancer type, querying the expert opinion database and the guideline excerpt database to determine a plurality of expert opinion entries and a plurality of guideline excerpts to form a plurality of expert opinion and guideline excerpt pairings. As an illustrative example, the plurality of expert opinion and guideline excerpt pairings correspond to the tumor type and the cancer type. In some embodiments, the method includes an additional operation of, generating instructions that cause rendering of the plurality of expert opinion and guideline excerpt pairings in addition to parameter selection fields. Following the generations of the instructions, the method includes the operations of transmitting the instructions to the network device to cause rendering of a display illustrating the video file and one of the transcript of the video file or the first guideline excerpt.

II. Terminology

In the following description, certain terminology is used to describe features of the invention. For example, in certain situations, the terms “logic” and “component” are representative of hardware, firmware or software that is configured to perform one or more functions. As hardware, logic (or component) may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, the logic (or component) may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the logic (or component) may be stored in persistent storage.

The term “network device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a desktop computer, a netbook, a medical device, or any general-purpose or special-purpose, user-controlled electronic device); a mainframe; a router; or the like.

A “communication” generally refers to information transmitted in one or more electrical signals that collectively represent electrically stored data in a prescribed format. Each communication may be in the form of one or more packets, frames, HTTP-based transmissions, signals transmitted over telephone lines, or any other series of bits having the prescribed format. The term “computerized” generally represents that any corresponding operations are conducted by hardware in combination with software and/or firmware.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

III. General Architecture

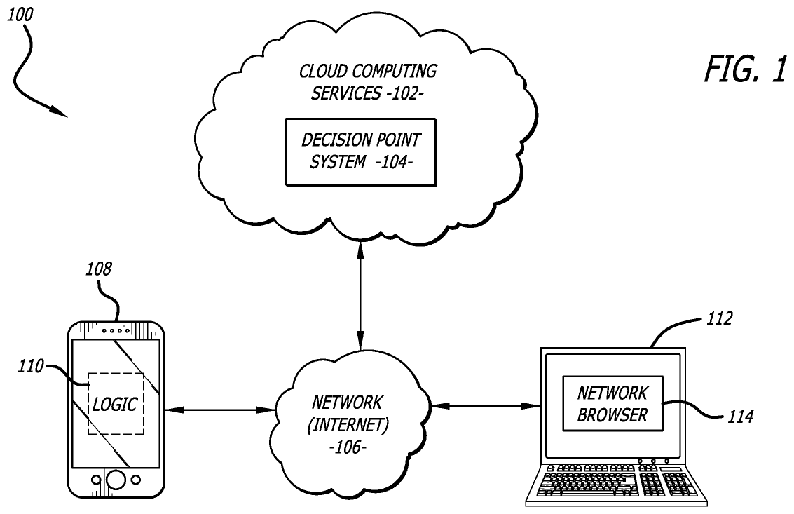

Referring to FIG. 1, is an exemplary system diagram illustrating communications between a decision point system and a plurality of network devices is shown according to some embodiments. FIG. 1 illustrates a networking environment 100 that includes a decision point system 104 operating on cloud computing services 102. FIG. 1 also includes network devices 108 and 112 that connected to a network 106 (e.g., the internet). The network 106 enables communicative coupling between the network devices 108, 112 and the decision point system 104 operating on the cloud computing services 102.

A decision point mobile application 108 may be installed on and configured for execution by a processor of the network device 108. Specifically, upon execution, the decision point mobile application 108 may be configured to initiate a communication link with the decision point system 104. Additionally, the decision point system 104 may be accessible via a web site, such as a web site loaded by the network browser 114 processing on the network device 112.

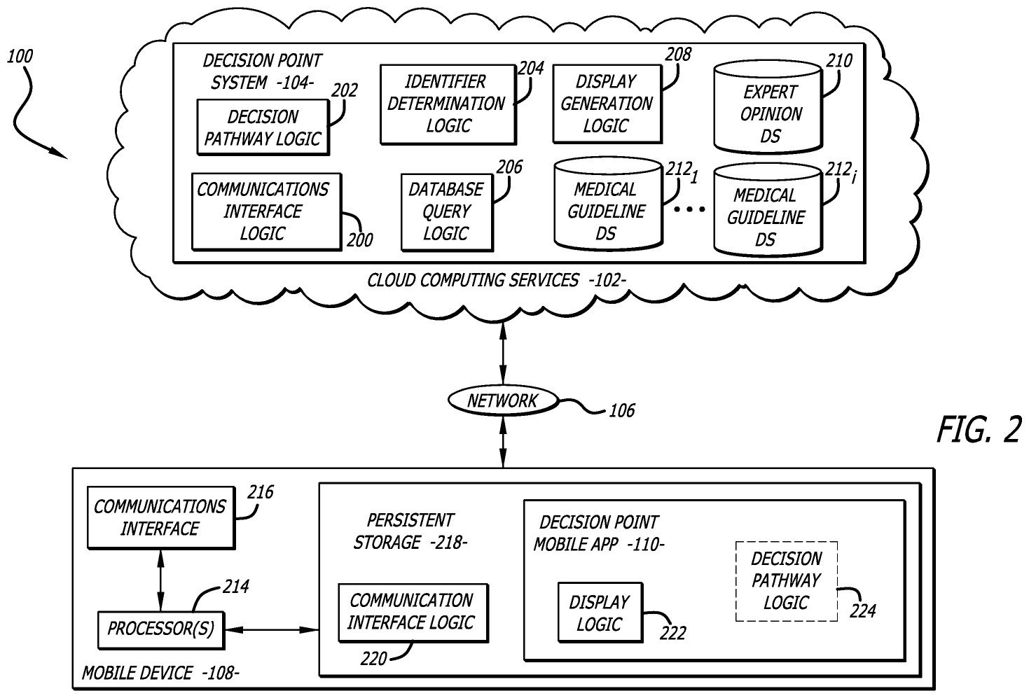

Referring now to FIG. 2, an exemplary embodiment of logical representations of the decision point system operating in cloud computing services and a decision point mobile application operating on a mobile device is shown according to some embodiments. Referring to the decision point system 104, in one embodiment, the system 104 may be stored on a non-transitory computer-readable storage medium of a server device operating within cloud computing services 102. The server device may include a housing, which is made entirely or partially of a hardened material (e.g., hardened plastic, metal, glass, composite or any combination thereof) that protects the circuitry within the housing, namely one or more processor(s), not shown, that are coupled to a communication interface, not shown. The communication interface, under control by a communication interface logic 200, enables communications with external network devices, such as the mobile device 108. According to one embodiment of the disclosure, the communication interface may be implemented as a physical interface including one or more ports for wired connectors. Additionally, or in the alternative, the communication interface may be implemented with one or more radio units for supporting wireless communications with other electronic devices. The communication interface logic 200 may perform operations of receiving and transmitting electronic data via the communication interface.

The non-transitory computer-readable storage medium of the server device may include some or all of the following components: a decision pathway logic 202, an identifier determination logic 204, a database query logic 206, a display generation logic 208, an expert opinion database 210, one or more medical guideline databases 212₁-212ᵢ (wherein i≥1) and the communication interface logic 200. Of course, when implemented as hardware, one or more of these logic units could be implemented separately from each other. Further, the one or more medical guideline databases 212₁-212ᵢ may implemented as a single database. In some embodiments, one or more of the databases may be stored remotely and accessible by the decision point system 104. Of course, one or more of the data stores may be implemented together.

In addition, FIG. 2 also illustrates an exemplary embodiment of a logical representation of the decision point mobile application 110. The decision point mobile application 110, in one embodiment, may be stored on a non-transitory computer-readable storage medium of an electronic device, i.e., the mobile device 108, that includes circuitry, namely one or more processor(s) 214 that are coupled to a communication interface 216. The communication interface 216, in combination with a communication interface logic 220, enables communications with external network devices, such as other network devices, not shown, as well as the network 106 and cloud computing services 102. The processor(s) (“processor”) 214 is further coupled to a persistent storage 218 and may have stored thereon the following logic as software modules: a display logic 222 and, optionally, a decision pathway logic 224. Additionally, although not shown, the decision point mobile application 110 may also include downloadable content that is available “offline” (i.e., when a network connection to the cloud computing services 102 is not available). The downloadable content may be at least a portion of one or more of (i) the expert opinion DS 210, and (ii) the medical guideline DS 212₁-212ᵢ In such an embodiment in which downloadable content is included with the decision point mobile application 110, the decision point mobile application 110 may also include logic similar to the identifier determination logic 204 and the database query logic 206. Referring to the downloadable content being “included” with the decision point mobile application 110 may refer to the downloadable content being stored on the persistent storage 218 and accessible by the decision point mobile application 110. Of course, it is contemplated that some or all of the logic modules of the decision point mobile application 110 may be implemented as hardware, and if so, such logic could be implemented separately from each other.

IV. Exemplary Methodologies

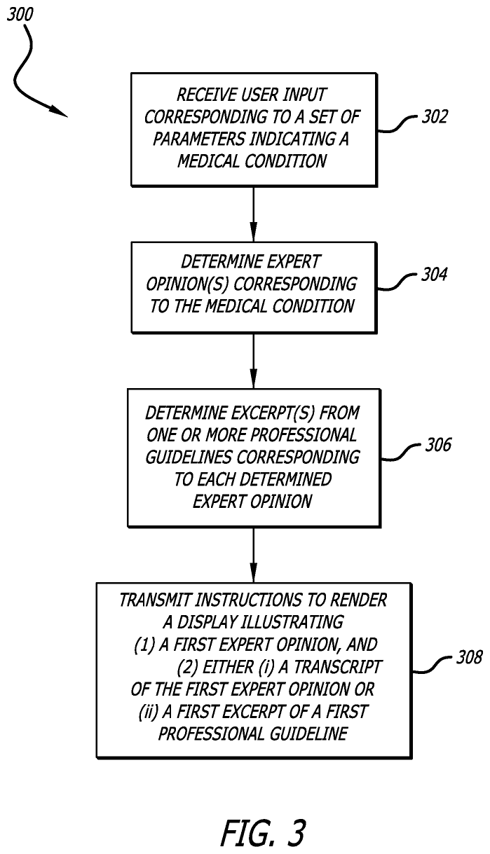

Referring now to FIG. 3, a flowchart illustrating a process of determining one or more expert opinions and one or more guideline excerpts to be rendered on a display screen as performed by the decision point system is shown according to some embodiments. Each block illustrated in FIG. 3 represents an operation performed in the method 300 of determining one or more expert opinions and one or more guideline excerpts to be rendered on a display screen based on received user input. In one embodiment, it is assumed that prior to the beginning of the method 300, a communication link has been initiated from a network device to the decision point system 104 of FIG. 1. In one embodiment, the initiation of a communication link may include processing of the decision point mobile application 110 by the mobile device 108. In a second embodiment, the initiation of a communication link may include loading a website via an internet browser that is configured to communicate with the decision point system 104. The initiation of the communication may cause the rendering of a display enabling a user to provide user input via one or more user input methodologies (e.g., buttons, checkboxes, radio buttons, text box, dropdown list, etc.). Thus, the method 300 commences when user input is received that corresponds to a set of parameters indicating a medical condition (block 302).

Examples of parameters may include, but are not limited or restricted to, a tumor type, a cancer type, a cancer stage, a molecular test indicator, a tumor extent indicator, a tumor stage, a metastasis level indicator, a biomarker, etc. More generally, the parameters may represent medical conditions that contribute to the determination of either a medical diagnosis or a treatment plan for a medical diagnosis. In other embodiments, the parameters may correspond to heart conditions and include, but are not limited or restricted to, blood pressure, cholesterol levels, history of smoking, family history of heart disease, whether the patient is diabetic, body fat percentage, etc. In yet other embodiments, the parameters may correspond to strokes and include, but are not limited or restricted to, blood pressure, history of smoking (e.g., and in combination with oral contraceptives), family history of strokes, gender, ethnicity, etc.

In yet other embodiments, the parameters may correspond to diabetes and include, but are not limited or restricted to, body weight, body fat percentage, age, level/frequency at which the patient exercises, characteristics of the patient's diet, etc. In yet other embodiments, the parameters may correspond to Alzheimer's disease and other dementias and include, but are not limited or restricted to, age, family history of Alzheimer's disease or dementia, presence of an existing mild cognitive impairment, whether the patient has Down syndrome, characteristics of the patient's lifestyle (e.g., diet, exercise, sleep), gender, the presence of previous head trauma, whether the patient engages with others, etc.

Responsive to receiving the user input, the decision point system 104 performs operations that determine one or more expert opinions that correspond to the medical condition (block 304). For instance, the operations performed may include parsing the set of parameters to determine one or more identifiers corresponding to the medical issue, wherein the identifiers may include a selected/input parameter via the user input (e.g., an identifier may include text corresponding to a selected radio button). For example, when user input options displayed on the display screen of the network device correspond to a tumor extent (e.g., T1, T2, T3, T4), wherein a radio button corresponds to each tumor extent option, the identifier may be the selected tumor extent option (e.g., “T3”).

The decision point system 104 then utilizes the identifier to query an expert opinion database to retrieve a first expert opinion entry corresponding to the selected parameter (or more generally, the medical issue). In some embodiments, the first expert opinion entry includes at least (i) a video file, (ii) a transcript of the video file and (iii) metadata. The metadata may provide information that correlates the expert opinion entry to one or more guideline excerpts.

Following the determination of one or more expert opinions, the decision point system 104 performs operations that determine one or more excerpts from one or more professional guidelines corresponding to each determined expert opinion (block 306). In one embodiment, the decision point system 104 utilizes metadata from each expert opinion entry to query one or more professional guideline excerpt databases, wherein each professional guideline excerpt database stores excerpts from a particular professional guideline (e.g., the NCCN guidelines referenced above). For instance, the decision point system 104 obtains metadata included with a first retrieved expert opinion entry (e.g., providing information on treatment for advanced NSCLC patients with common activating EGFR mutation). The decision point system 104 then queries a first professional guideline excerpt database (e.g., a database storing excerpts from the NCCN guidelines) and retrieves one or more corresponding guideline excerpts (e.g., a first retrieved excerpt may be the NCCN guideline on “EGFR mutation discovered before first-line therapy”).

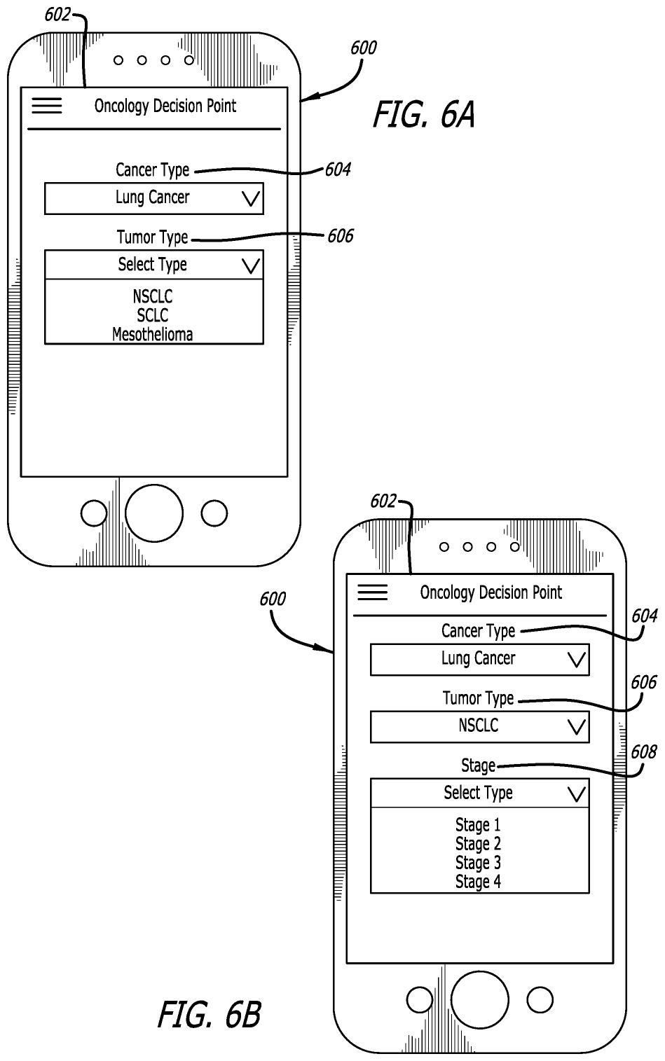

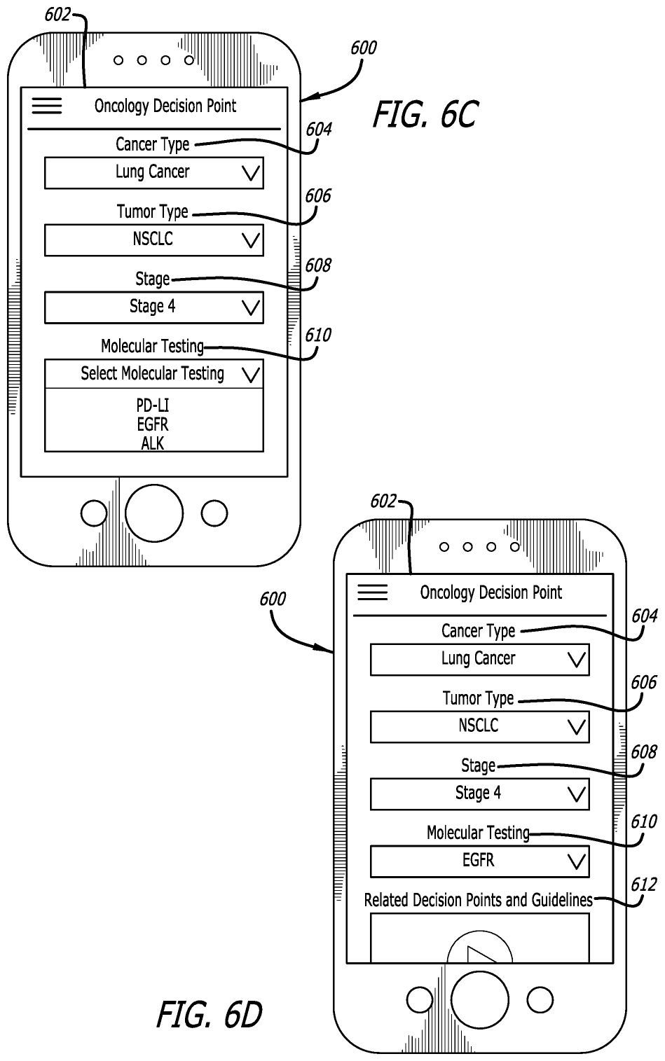

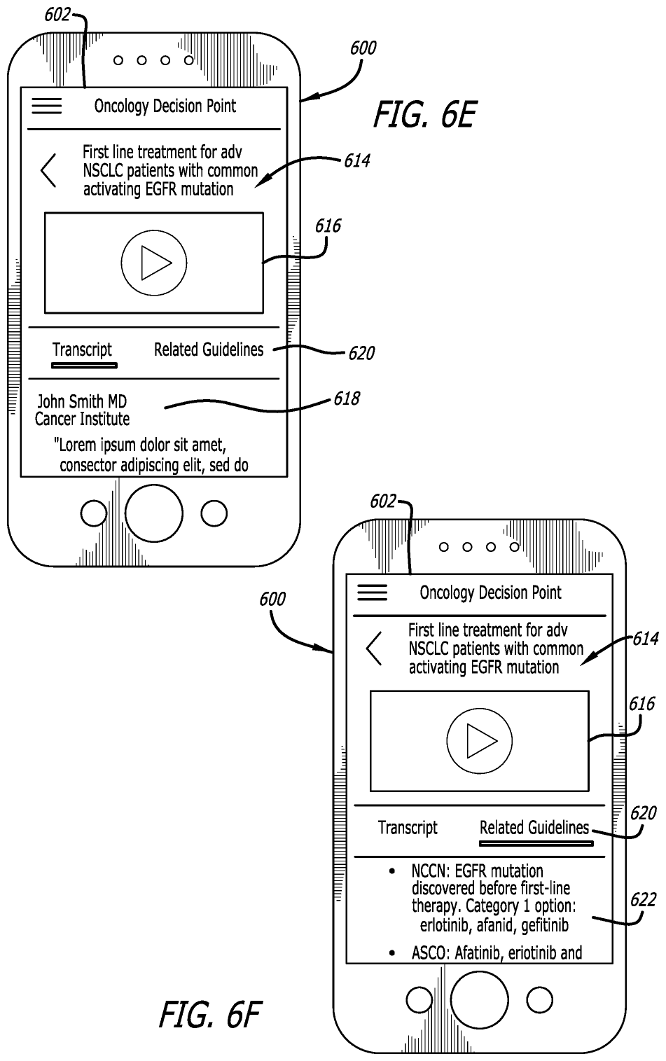

Following the querying of the expert opinion database and the one or more professional guideline excerpt databases and retrieval of one or more expert opinion entries and one or more guideline excerpts, the decision point system 104 generates instructions to cause rendering of a display on a display screen of the network device via the communication link and transmits the instructions. More specifically, the decision point system 104 determines a type of display screen of the network device (e.g., mobile phone display screen, tablet display screen, laptop or other computer screen, etc.) in order to generate instructions that will cause rendering of a display that is proportionate to and accurately sized for the display screen. Based on the determined display screen, instructions are generated in order to cause rendering of a display that provides quick access to (1) the expert opinion video file, and (2) either (i) the transcript of the expert opinion video file, or (ii) a first professional guideline excerpt. For example, referring to the display for a display screen of a mobile phone, a title of the expert opinion may be provided above an embedded video player, which is provided above either the video transcript or text of the related guideline. Exemplary examples of such a display are illustrated in FIGS. 6E-6F. In some embodiments, a similar display may be generated for a display screen of a laptop or other computer (or any other network device). As an alternative embodiment, the instructions may cause a display to be rendered wherein the video player and the transcript and/or text of the guideline excerpt are provided in a side-by-side manner. Other formatting options has been contemplated and the disclosure is not intended to be limited to the illustrative examples specifically discussed.

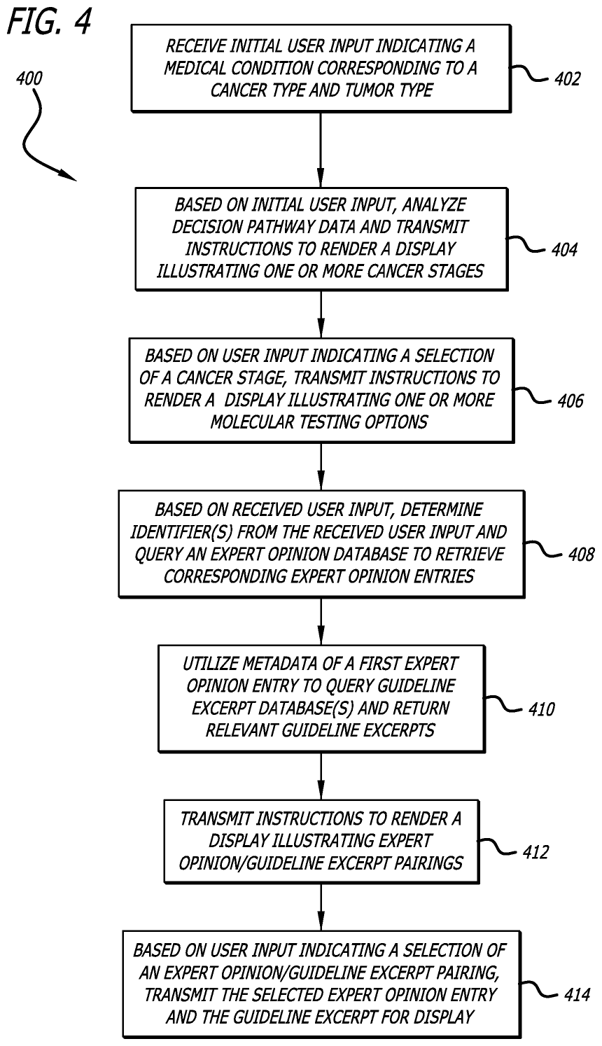

Referring now to FIG. 4, a flowchart illustrating a first embodiment of a detailed process of determining one or more expert opinions and one or more guideline excerpts to be rendered on a display screen as performed by the decision point system is shown. Each block illustrated in FIG. 4 represents an operation performed in the method 400 of determining one or more expert opinions and one or more guideline excerpts to be rendered on a display screen based on received user input according to a first embodiment. In some embodiments, the same assumptions discussed above with respect to FIG. 3 may also apply with respect to the discussion of FIG. 4.

The method 400 commences when the decision point system 104 of FIG. 1 receives initial user input indicating a medial issue corresponding to a cancer type and a tumor type (block 402). As discussed above, the input indicating a cancer type provides a first step in filtering through all expert opinions stored within the expert opinion database (e.g., a first step in traversing a tree structure). After filtering based on the cancer type, the remaining expert opinion entries may be referred to as a “cancer specific subset of expert opinion entries.” Additionally, the cancer type provides a second step in which the cancer specific subset of expert opinions are filtered (e.g., a second step in traversing a tree structure). After filtering based on the tumor type, the remaining expert opinion entries may be referred to as a “cancer-tumor specific subset of expert opinion entries.”

Based on the received initial user input, the decision point system 104 analyzes decision pathway data, as discussed above, and transmits instructions to a network device used by the user providing input, wherein the instructions, when executed, cause the rendering of a display that illustrates one or more cancer stage options that correspond to the cancer type and tumor type (block 404). As an example, FIGS. 6A-6B illustrate the change in a display rendered on the display screen of a network device based on the selection of a tumor type (and implicitly in combination with the selection of the cancer type). Although described herein as the execution of the instructions causing the rendering of a display, the execution of the instructions may also cause the modification of a portion of the display based on the programming languages used in constructing the website or mobile application.

Based on the received user input indicating a selection of a cancer stage, the decision point system 104 further traverses the decision pathway data and transmits instructions to the network device causing the rendering of a display that illustrates molecular testing options that correspond to the cancer stage (block 406). Based on the received user input, the decision point system 104 determines one or more identifiers from the user input, as discussed above, and queries an expert opinion database using the one or more identifiers to retrieve one or more corresponding expert opinion entries (block 408).

Subsequent to the retrieval of the one or more corresponding expert opinion entries, the decision point system 104 utilizes the metadata included in a first expert opinion entry to query one or more guideline excerpt databases to retrieve one or more guideline excerpts that correspond to the set of parameters included in the received user input (block 410). At this stage, the decision point system 104 may form “expert opinion and guideline excerpt pairings,” wherein a first expert opinion and guideline excerpt pairing is comprised of: (i) a first expert opinion entry, and (ii) a first guideline excerpt that corresponds to the first expert opinion.

Following the querying of the expert opinion database and the one or more professional guideline excerpt databases and retrieval of one or more expert opinion entries and one or more guideline excerpts, the decision point system 104 generates instructions to cause rendering of a display on a display screen of the network device and transmits the instructions to the network device (block 412). Specifically, the display provides access to one or more expert opinion and guideline excerpt pairings, including, for each expert opinion and guideline excerpt pairing: (1) an expert opinion video file, and (2) either (i) a transcript of the expert opinion video file, or (ii) a guideline excerpt that corresponds to the expert opinion video file, as discussed above. The instructions are then transmitted to a network device. Further, based on received user input indicating the selection of an expert opinion and guideline excerpt pairing, the decision point system 104 transmits the selected expert opinion entry (e.g., the video file and the text of the transcript) and guideline excerpt (e.g., text) for display (block 414).

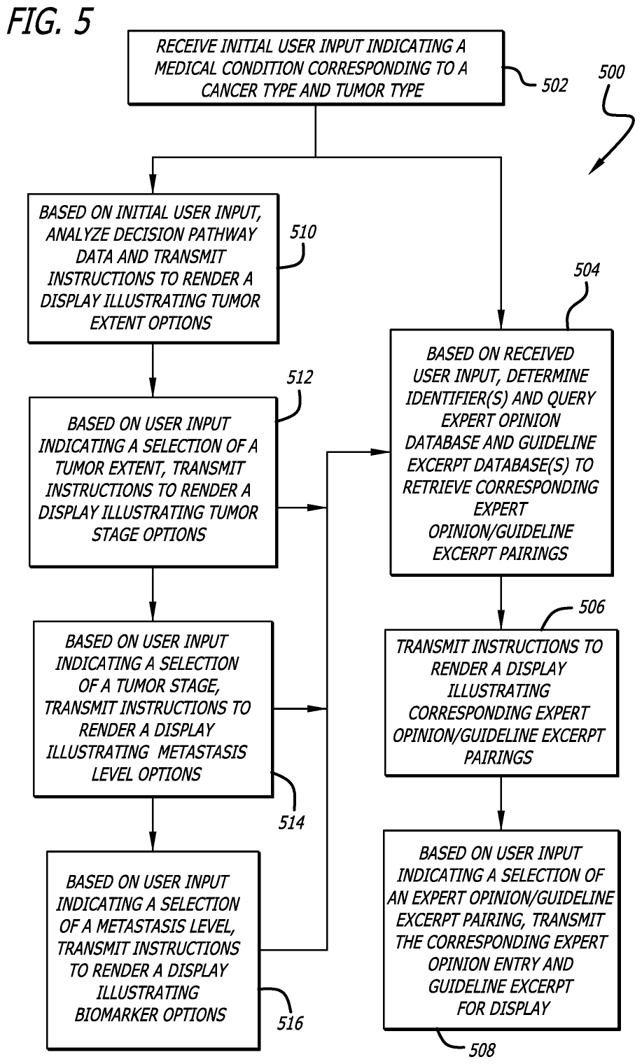

Referring now to FIG. 5, a flowchart illustrating a second embodiment of a detailed process of determining one or more expert opinions and one or more guideline excerpts to be rendered on a display screen as performed by the decision point system is shown. Each block illustrated in FIG. 5 represents an operation performed in the method 500 of determining one or more expert opinions and one or more guideline excerpts to be rendered on a display screen based on received user input according to a second embodiment. In some embodiments, the same assumptions discussed above with respect to FIG. 3 may also apply with respect to the discussion of FIG. 5. The method 500 commences when the decision point system 104 of FIG. 1 receives initial user input indicating a medical condition corresponding to a cancer type and a tumor type as discussed above (block 502).

Based on received user input, determine one or more identifiers and query (i) an expert opinion database, and (ii) one or more guideline excerpt databases, to retrieve expert opinion entries and guideline excerpts that correspond to the received user input (block 504). The decision point system 104 may then form expert opinion and guideline excerpt pairings.

Following the querying of the expert opinion database and the one or more professional guideline excerpt databases and retrieval of one or more expert opinion entries and one or more guideline excerpts, the decision point system 104 generates instructions to cause rendering of a display on a display screen of a network device used by the user providing input and transmits the instructions to the network device (block 506). Details regarding embodiments of the display are discussed above with respect to FIG. 4. Additionally, based on received user input indicating a selection of an expert opinion and guideline excerpt pairing, the decision point system 104 transmits the selected expert opinion entry (e.g., the video file and the text of the transcript) and guideline excerpt for display (block 508).

As FIG. 5 illustrates, additional operations may be performed by the decision point system 104 in parallel (e.g., simultaneously or concurrently, i.e., at least partially overlapping in time) (blocks 510-516). For example, based on received initial user input corresponding to a selection of a cancer type and a tumor type, the decision point system 104 may filter through the expert opinion database as discussed above (e.g., analyze decision pathway data) and transmit instructions to the network device of the user, wherein the instructions, when executed, cause the rendering of a display that illustrates one or more tumor extent options corresponding to the cancer type and the tumor type (block 510). After filtering based on the cancer type and the tumor type, the remaining expert opinion entries may be referred to as a “cancer-tumor specific subset of expert opinion entries.”

Upon receipt of user input corresponding to a selection of a tumor extent, the decision point system 104 may filter through the cancer-tumor specific subset of expert opinion entries according to the selected tumor extent option and transmit instructions to the network device of the user, wherein the instructions, when executed, cause the rendering of a display that illustrates one or more tumor stage options corresponding to the selected tumor extent (block 512). After filtering based on the tumor extent, the remaining expert opinion entries may be referred to as a “cancer-tumor-tumor extent specific subset of expert opinion entries.” Additionally, the received user input corresponding to a selection of a tumor extent may be utilized by the decision point system 104 to determine expert opinion and guideline excerpt pairings from the cancer-tumor-tumor extent specific subset of expert opinion entries and proceed from block 504 as discussed above.

Upon receipt of user input corresponding to a selection of a tumor stage, the decision point system 104 may filter through the cancer-tumor-tumor stage specific subset of expert opinion entries and transmit instructions to the network device of the user, wherein the instructions, when executed, cause the rendering of a display that illustrates one or more metastasis level options corresponding to the selected tumor stage (block 514). After filtering based on the tumor stage, the remaining expert opinion entries may be referred to as a “cancer-tumor-tumor extent-tumor stage specific subset of expert opinion entries.” Additionally, the received user input corresponding to a selection of a tumor stage option may be utilized by the decision point system 104 to determine expert opinion and guideline excerpt pairings from the cancer-tumor-tumor extent-tumor stage specific subset of expert opinion entries and proceed from block 504 as discussed above.

Upon receipt of user input corresponding to a selection of a metastasis level, the decision point system 104 may filter through the cancer-turn or-turn or extent-tumor stage specific subset of expert opinion entries and transmit instructions to the network device of the user, wherein the instructions, when executed, cause the rendering of a display that illustrates one or more biomarker options corresponding to the selected metastasis level (block 516). Additionally, the received user input corresponding to a selection of a metastasis level may be utilized by the decision point system 104 to determine expert opinion and guideline excerpt pairings from the cancer-tumor-tumor extent-tumor stage metastasis level specific subset of expert opinion entries and proceed from block 504 as discussed above.

V. Illustrative Embodiment

Referring now to FIGS. 6A-6F, are illustrations of display screens an exemplary embodiment of the detailed process of FIG. 4 shown. Each of the illustrations presented in FIGS. 6A-6F provide an exemplary display screen 602 of a network device 600 having stored thereon the decision point mobile application 110 of FIG. 1, wherein the decision point mobile application 110, upon execution, has initiated a communication, link with the decision point system 104. The embodiment illustrated provides an example of an oncology-directed aspect of the decision point system 104; however, as discussed above, the decision point system 104 applies to numerous other areas of medicine. With respect to FIGS. 6A-6F, it is assumed that the decision point mobile application 110 has initiated a communication link and is in communication with the decision point system 104. Prior to FIG. 6A, user input has been received indicating a cancer type 604 of “lung cancer.”

Referring now to FIG. 6A, based on the received user input indicating a cancer type 604 of “lung cancer,” the decision point system 104 has provided the decision point mobile application 110 with instructions that, upon execution, caused the rendering of user input options corresponding to a tumor type 606. Referring to FIG. 6B, user input has been received by the decision point mobile application 110 and provided to the decision point system 104 indicating a selection of a tumor type 606 of “NSCLC” (Non-small-cell lung carcinoma). Based on the received user input indicating a tumor type 606 of “NSCLC,” the decision point system 104 has provided the decision point mobile application 110 with instructions that, upon execution, caused the rendering of user input options corresponding to a tumor stage 606.

Referring to FIG. 6C, user input has been received by the decision point mobile application 110 and provided to the decision point system 104 indicating a selection of a tumor stage 608 of “stage 4.” Based on the received user input indicating a tumor stage 608 of “stage 4,” the decision point system 104 has provided the decision point mobile application 110 with instructions that, upon execution, caused the rendering of user input options corresponding to molecular testing option 610. Referring to FIG. 6D, user input has been received by the decision point mobile application 110 and provided to the decision point system 104 indicating a selection of a molecular testing option 610 of “EGFR” (Epidermal Growth Factor Receptor) Based on the received user input indicating a molecular testing option 610 of “EGFR,” the decision point system 104 has provided the decision point mobile application 110 with instructions that, upon execution, caused the rendering of one or more expert opinion and guideline excerpt pairings 612 as discussed in FIG. 4.

Referring to FIG. 6E, user input has been received by the decision point mobile application 110 and provided to the decision point system 104 indicating a selection of a first expert opinion and guideline excerpt pairing 614. Based on the received user input indicating selection of the first expert opinion and guideline excerpt pairing 614, the decision point system 104 has provided the decision point mobile application 110 with instructions that, upon execution, caused the rendering of a display of the first expert opinion and guideline excerpt pairing 614. As seen, the display of the first expert opinion and guideline excerpt pairing 614 includes an embedded video 616 from the corresponding expert opinion entry and a transcript 618 of the video 616. Additionally, a selection bar 620 provides the ability for a user to select from viewing the transcript or related guideline excerpts 622, as seen in FIG. 6F.

Referring now to FIG. 6F, user input has been received by the decision point mobile application 110 via the selection bar 620 resulting in the display of the related guideline excerpts 622 that correspond to the video 616. The selection bar 620 provides the ability to toggle between viewing the transcript 618 of the video 616 and the related guideline excerpts 622. It should be noted that in some instances, only a single guideline excerpt corresponding to the video 616 may be available. In yet other embodiments, no guideline excerpts corresponding to the video 616 may be available (e.g., the content of video 616 is so new that corresponding material has not been added to the professional guidelines).

In the foregoing description, the invention is described with reference to specific exemplary embodiments thereof. It will, however, be evident that various modifications and changes may be made thereto without departing from the broader spirit and scope of the invention as set forth in the appended claims.

What is claimed is:

1. A method of determining and causing display of particularized medical information comprising:
receiving user input corresponding to a set of parameters indicating a medical condition;
parsing the set of parameters to determine one or more identifiers corresponding to the medical condition;
querying an expert opinion database according to the one or more identifiers to retrieve a first expert opinion entry corresponding to the medical condition, wherein the first expert opinion entry includes (i) a video file, (ii) a transcript of the video file and (iii) metadata;
querying a guideline excerpt database according to the metadata to retrieve a first guideline excerpt corresponding to the first expert opinion; and
transmitting instructions, to a network device, to cause rendering of a display illustrating the video file and one of the transcript of the video file or the first guideline excerpt.

2. The method of claim 1, wherein the medical condition is related to cancer, and the set of parameters are indicative of a medical diagnosis of the cancer.

3. The method of claim 2, wherein the set of parameters include a tumor type, a cancer type, and at least one of (i) a cancer stage, (ii) a molecular test indicator, (iii) a tumor extent indicator, (iv) a tumor stage, (v) a metastasis level indicator, or (vi) a biomarker.

4. The method of claim 3, wherein the guideline excerpt database includes excerpts from at least one of the National Comprehensive Cancer Network (NCCN) guidelines, the American Society of Clinical Oncology (ASCO) guidelines or the European Society for Medical Oncology (ESMO) guidelines.

5. The method of claim 1, further comprising:
responsive to receiving initial user input that includes the tumor type and the cancer type, querying the expert opinion database and the guideline excerpt database to determine a plurality of expert opinion entries and a plurality of guideline excerpts to form a plurality of expert opinion and guideline excerpt pairings, wherein the plurality of expert opinion and guideline excerpt pairings correspond to the tumor type and the cancer type;
generating second instructions that cause rendering of the plurality of expert opinion and guideline excerpt pairings in addition to parameter selection fields; and
transmitting instructions, to the network device, to cause rendering of a display illustrating the video file and one of the transcript of the video file or the first guideline excerpt.

6. The method of claim 1, wherein the metadata indicates a link between the first expert opinion entry and one or more guideline excerpts including the first guideline excerpt.

7. The method of claim 1, wherein the expert opinion database and the guideline excerpt database are stored in cloud computing services.

8. A non-transitory computer readable storage medium having stored thereon instructions, the instructions being executable by one or more processors to perform operations comprising:
receiving user input corresponding to a set of parameters indicating a medical condition;
parsing the set of parameters to determine one or more identifiers corresponding to the medical condition;
querying an expert opinion database according to the one or more identifiers to retrieve a first expert opinion entry corresponding to the medical condition, wherein the first expert opinion entry includes (i) a video file, (ii) a transcript of the video file and (iii) metadata;
querying a guideline excerpt database according to the metadata to retrieve a first guideline excerpt corresponding to the first expert opinion; and
transmitting instructions, to a network device, to cause rendering of a display illustrating the video file and one of the transcript of the video file or the first guideline excerpt.

9. The non-transitory computer readable storage medium of claim 8, wherein the medical condition is related to cancer, and the set of parameters are indicative of a medical diagnosis of the cancer.

10. The non-transitory computer readable storage medium of claim 9, wherein the set of parameters include a tumor type, a cancer type, and at least one of (i) a cancer stage, (ii) a molecular test indicator, (iii) a tumor extent indicator, (iv) a tumor stage, (v) a metastasis level indicator, or (vi) a biomarker.

11. The non-transitory computer readable storage medium of claim 10, wherein the guideline excerpt database includes excerpts from at least one of the National Comprehensive Cancer Network (NCCN) guidelines, the American Society of Clinical Oncology (ASCO) guidelines or the European Society for Medical Oncology (ESMO) guidelines.

12. The non-transitory computer readable storage medium of claim 8, wherein the instructions being executable by the one or more processors to perform further operations comprising:
responsive to receiving initial user input that includes the tumor type and the cancer type, querying the expert opinion database and the guideline excerpt database to determine a plurality of expert opinion entries and a plurality of guideline excerpts to form a plurality of expert opinion and guideline excerpt pairings, wherein the plurality of expert opinion and guideline excerpt pairings correspond to the tumor type and the cancer type;
generating second instructions that cause rendering of the plurality of expert opinion and guideline excerpt pairings in addition to parameter selection fields; and
transmitting instructions, to the network device, to cause rendering of a display illustrating the video file and one of the transcript of the video file or the first guideline excerpt.

13. The non-transitory computer readable storage medium of claim 8, wherein the metadata indicates a link between the first expert opinion entry and one or more guideline excerpts including the first guideline excerpt.

14. A system for automatically determining and displaying particularized medical information, the system comprising:
a processor; and
a memory communicatively coupled to the processor, the memory includes:
identifier determination logic configured to, upon execution by the processor, parse user input to determine one or more identifiers corresponding to a medical condition,
decision pathway logic, configured to, upon execution by the processor, analyze a predetermined decision pathway in accordance with the one or more identifiers and determine one or more parameter selection fields to be provided to a user that further particularize the medical condition;
database query logic configured to, upon execution by the processor, (1) query an expert opinion database according to the one or more identifiers to retrieve a first expert opinion entry corresponding to the medical condition, wherein the first expert opinion entry includes (i) a video file, (ii) a transcript of the video file and (iii) metadata, and (2) query a guideline excerpt database according to the metadata to retrieve a first guideline excerpt corresponding to the first expert opinion, and
display generation logic configured to, upon execution by the processor, generate instructions, to be executed by a network device, to cause rendering of a display illustrating the video file and one of the transcript of the video file or the first guideline excerpt.

15. The system of claim 14, wherein the medical condition is related to cancer, and the set of parameters are indicative of a medical diagnosis of the cancer.

16. The system of claim 15, wherein the set of parameters include a tumor type, a cancer type, and at least one of (i) a cancer stage, (ii) a molecular test indicator, (iii) a tumor extent indicator, (iv) a tumor stage, (v) a metastasis level indicator, or (vi) a biomarker.

17. The system of claim 16, wherein the guideline excerpt database includes excerpts from at least one of the National Comprehensive Cancer Network (NCCN) guidelines, the American Society of Clinical Oncology (ASCO) guidelines or the European Society for Medical Oncology (ESMO) guidelines.

18. The system of claim 14, further comprising:
responsive to receiving initial user input that includes the tumor type and the cancer type, querying the expert opinion database and the guideline excerpt database to determine a plurality of expert opinion entries and a plurality of guideline excerpts to form a plurality of expert opinion and guideline excerpt pairings, wherein the plurality of expert opinion and guideline excerpt pairings correspond to the tumor type and the cancer type;
generating second instructions that cause rendering of the plurality of expert opinion and guideline excerpt pairings in addition to parameter selection fields; and
transmitting instructions, to the network device, to cause rendering of a display illustrating the video file and one of the transcript of the video file or the first guideline excerpt.

19. The system of claim 20, wherein the metadata indicates a link between the first expert opinion entry and one or more guideline excerpts including the first guideline excerpt.

20. The system of claim 14, wherein the processor and the memory correspond to cloud computing services.
